(19) United States
(12) Patent Application Publication
(10) Pub. No.: US 2010/0331410 A1
(43) Pub. Date: Dec. 30, 2010

(54) Biaryl Amides

(75) Inventors: Robert Coleman, Cornwall (GB); David Middlemiss, Hertfordshire (GB)

Correspondence Address: Jonathan P. O''Brien, Ph.D.;Honigman Miller Schwartz and Cohn LLP, 350 East Michigan Avenue, Suite 300, KALAMAZOO, MI, 49007, US

(73) Assignee: Asterand UK Limited, Hertfordshire (GB)

(21) Appl. No.: 12/865,321

(22) PCT Filed: Feb. 5, 2009
(86) PCT No.: PCT/GB2009/000317

§ 371 Date: Jul. 29, 2010

(30) Foreign Application Priority Data
Feb. 5, 2008 (GB) 0802104.0
Feb. 5, 2008 (GB) 0802105.7
Feb. 5, 2008 (GB) 0802106.5
Feb. 5, 2008 (GB) 0802107.3

(51) Int. Cl. A61K 31/24; A61K 31/167; A61P 27/02; A61P 27/06; C07C 229/00; C07C 235/24

(52) U.S. Cl. 514/539; 514/616; 560/45; 564/158

(57) ABSTRACT

A compound of formula:

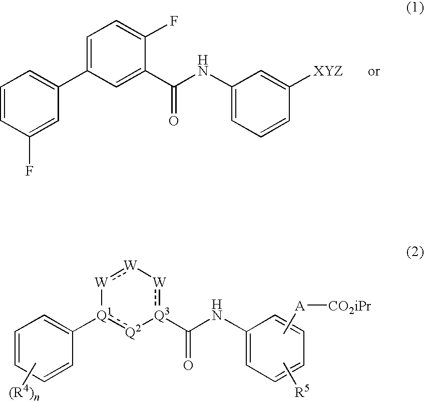

for treating ocular hypertension.

### FIELD OF THE INVENTION

The present invention relates to compounds, pharmaceutical compositions comprising these compounds, and their use in the treatment of glaucoma and ocular hypertension.

### BACKGROUND OF THE INVENTION

Ocular hypotensive agents are useful in the treatment of a variety of ocular hypertensive conditions, including post-surgical and post-laser trabeculectomy ocular hypertensive episodes, glaucoma and as pre-surgical adjunctive treatment.

Glaucoma is a disease of the eye characterized by increased intraocular pressure. On the basis of its etiology, glaucoma has been classified as primary or secondary. For example, primary glaucoma in adults (congenital glaucoma) may be either open angle or acute or chronic angle-closure. Secondary glaucoma results from pre-existing ocular diseases such as uveitis, intraocular tumor or enlarged cataract.

The underlying causes of glaucoma are not known. The increased intraocular pressure is due to the obstruction of aqueous humour outflow. In chronic open-angle glaucoma, the anterior chamber and its anatomic structures appear normal, but drainage of the aqueous humour is impeded. In acute or chronic angle-close glaucoma, the anterior chamber is shallow, the filtration angle is narrowed, and the iris may obstruct the trabecular meshwork at the entrance of the canal of Schlemm. Dilation of the pupil may push the root of the iris forward against the angle, and may produce papillary block and thus precipitate an acute attack. Eyes with narrow anterior chamber angles are predisposed to acute angle-closure glaucoma attacks of various degrees of severity.

Secondary glaucoma is caused by any interference with the flow of aqueous humour from the posterior chamber into the anterior chamber and consequently into the canal of Schlemm. Inflammatory disease of the anterior segment may prevent aqueous escape by causing complete posterior synechia in iris bombe and may plug the drainage channel with exudates. Other common causes are intraocular tumors, enlarged cataracts, central retinal vein occlusion, trauma to the eye, operative procedure and intraocular hemorrhage.

Considering all types together, glaucoma occurs in approximately 2% of all persons over the age of 40 and may be asymptomatic for years before progressing to rapid loss of vision.

In cases where surgery is not indicated, topical β-adrenergic antagonists have traditionally been the drugs of choice for treating glaucoma.

It has long been known that one of the sequalae of glaucoma is damage to the optic nerve head. This damage, referred to as “cupping”, results in depressions in areas of the nerve fibre of the optic disk. Loss of sight from cupping is progressive and can lead to blindness if the condition is not treated effectively.

Prostaglandins were earlier reported as potent ocular hypertensives; however, evidence accumulated in the last two decades shows that some prostaglandins are highly effective ocular hypotensive agents and are ideally suited to the long term medical management of glaucoma (see, for example, Starr, M. S. Exp. Eye Res 1971, 11, pp. 170-177; Bito, L. Z. Biological Protection with Prostaglandins Cohen, M. M. ed. Boca Raton, Fla., CRC Press Inc., 1985, pp 231-252; and Bito, L. Z. Applied Pharmacology in the Medical Treatment of Glaucomas Drance, S. M. and Neufled, A. H. eds., New York, Grune & Stratton, 1984, pp 477-505). Such prostaglandins include PGF_{2α}, PGF_{1α}, PGE₂ compounds.

Certain EP₂-receptor-selective prostaglandin E₂ agonists are disclosed in Paralkar V. M. et al, Proc. Nat. Acad. Sci. vol 100 pp 6736-6740, 2003.

Certain EP₂ agonists are disclosed in WO2005/080367. In particular, this publication discloses compounds falling within the general formula (I):

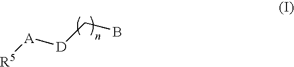

or a salt, solvate and chemically protected form thereof, wherein:
R⁵ is an optionally substituted C₅₋₂₀ aryl or C₄₋₂₀ alkyl group;
A is selected from the group consisting of:

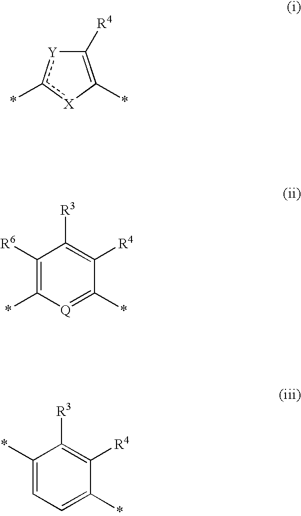

wherein X and Y are selected from the group consisting of: O and CR³; S and CR³; NH and CR³; NH and N; O and N; S and N; N and S; and N and O, and where the dotted lines indicate a double bond in the appropriate location, and where Q is either N or CH;
R³ is selected from H, F, Cl and optionally substituted C₁₋₄ alkyl, C₁₋₄ alkoxy, C₅₋₇ aryl and C₅₋₇ aryl-C₁₋₄ alkyl groups;
R⁴ is selected from H, F, Cl and optionally substituted C₁₋₄ alkyl, C₁₋₄ alkoxy, C₅₋₇ aryl and C₅₋₇ aryl-C₁₋₄ alkyl groups;
R⁶ is selected from H, F, Cl and optionally substituted C₁₋₄ alkyl, C₁₋₄ alkoxy, C₅₋₇ aryl and C₅₋₇ aryl-C₁₋₄ alkyl groups;
D is selected from:

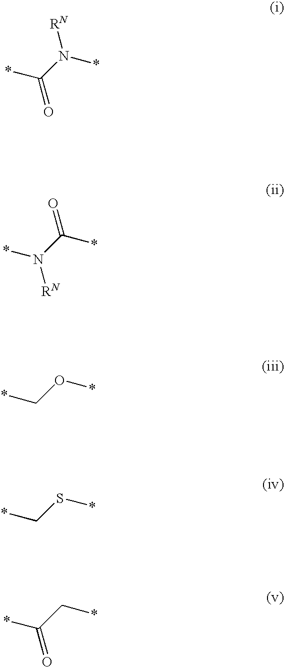

B is selected from the group consisting of:

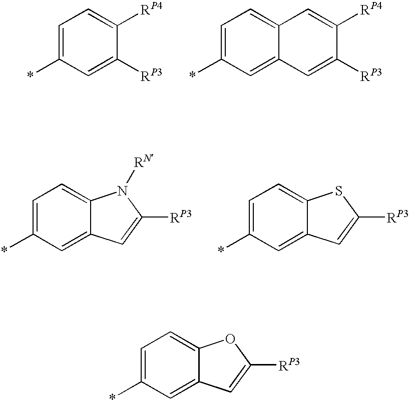

where R^{N′} is selected from H and C₁₋₄ alkyl;
where one of R^{P3} and R^{P4} is —Cₘ alkylene-R² and the other of R^{P3} and R^{P4} is H, m and n can be 0 or 1, and m+n=1 or 2; and additionally when R^{P3} is —Cₘ alkylene-R², m can also be 2 or 3, and m+n=1, 2, 3 or 4, and when R² is tetrazol-5-yl, m+n may be 0; or where one of R^{P3} and R^{P4} is —O—CH₂—R², and the other of R^{P3} and R^{P4} is H, n is 0;
R^{N} is H or optionally substituted C₁₋₄ alkyl;
R² is either:
(i) —CO₂H (carboxy);

### (ii) —CONH₂;

(iii) —CH₂—OH (methoxy); or
(iv) tetrazol-5-yl.

Amongst these compounds were:

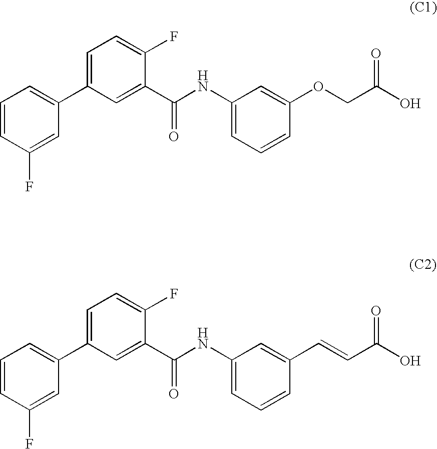

Current agents do not reduce IOP to normal levels or below and many are limited by their propensity to cause ocular hyperemia. There is thus a major unmet need for therapeutics that are more efficacious and safer.

### SUMMARY OF THE INVENTION

A first aspect of the invention comprises a compound of formula (1) or a pharmaceutically acceptable salt or solvate thereof for use in a method of therapy.

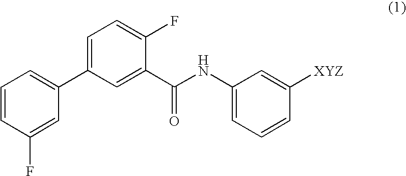

wherein:

### X is OCH₂, CH═CH or CH₂;

### Y is —CO₂ or —C(O)NH;

Z is a straight or branched chain alkyl group of 1-6 carbon atoms, a cycloalkyl group of 1-6 carbon atoms, either of which may be optionally substituted with one or more groups selected from OH, CO₂H, CONH₂, OR¹, CO₂R¹, CONHR¹ and OCO₂R¹;
R¹ is a straight or branched chain alkyl group of 1-6 carbon atoms optionally substituted with one or more groups selected from OH, CO₂H, CONH₂, OR², CO₂R² and CONHR²;
R² is selected from a straight or branched chain alkyl group of 1-6 carbon atoms optionally substituted with one or more groups independently selected from OH, CO₂H, CONH₂, OR³, CO₂R³ and CONHR³; and
R³ is a straight or branched chain alkyl group of 1-6 carbon atoms; or
YZ together form a group selected from

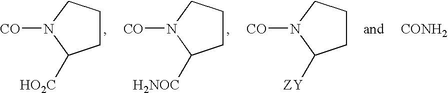

where Y and Z are as defined above.

A second aspect of the invention comprises a compound of formula (1) or a pharmaceutically acceptable salt or solvate thereof as defined in the first aspect, for use in the treatment of ocular hypertension.

A third aspect of the present invention provides a pharmaceutical composition comprising a compound of formula (1) or a pharmaceutically active acceptable salt or solvate thereof, as defined in the first aspect together with a pharmaceutically acceptable carrier.

A fourth aspect of the present invention provides a method of treating ocular hypertension which comprises administering to a mammal having ocular hypertension a therapeutically effective amount of a compound of the formula (1) or a pharmaceutically acceptable salt or solvate thereof, as defined in the first aspect.

A fifth aspect of the present invention provides a method of providing neuroprotection to the eye of a mammal which comprises administering to a mammal in need of neuroprotection a therapeutically effective amount of a compound of the formula (1) or a pharmaceutically acceptable salt or solvate thereof, as defined in the first aspect.

A sixth aspect of the present invention provides a contact lens or a contact lens solution comprising a compound of the formula (1) or a pharmaceutically acceptable salt or solvate thereof, as defined in the first aspect.

A seventh aspect of the invention comprises a compound of formula (1) or a pharmaceutically acceptable salt or solvate thereof as defined in the first aspect, with the proviso that the compound does not have the structure:

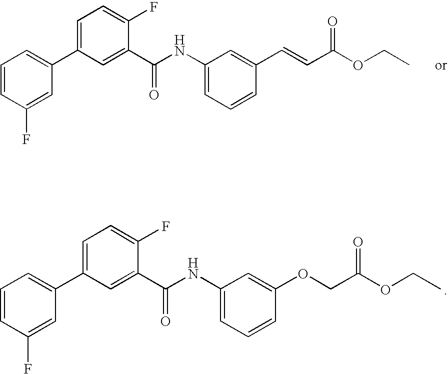

### Further Features of the First to Seventh Aspects of the Invention

In some embodiments, X is OCH₂. Thus in these embodiments, the compound is of formula (Ia):

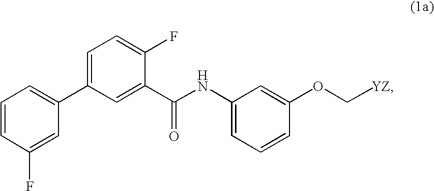

where Y and Y are as defined above.

Preferably Y is —CO₂.

Preferably, Z is a straight or branched chain alkyl group having 1, 2, 3 or 4 carbon atoms, most preferably 1, 2 or 3 carbon atoms.

Preferably, Z is selected from the group consisting of methyl, ethyl, n-propyl, iso-propyl, n-butyl, sec-butyl, iso-butyl, tert-butyl and n-pentyl. In some embodiments, Z is selected from methyl, n-propyl, iso-propyl, n-butyl, sec-butyl, iso-butyl, tert-butyl and n-pentyl. In further embodiments, Z is selected from methyl and iso-propyl.

Preferably, where Z is substituted, it is substituted with 1 or 2 groups selected from OH, CO₂H, CONH₂, OR¹, CO₂R¹ and CONHR¹. In some embodiment, Z is substituted with a single OH group. In other embodiments, Z is unsubstituted.

Where Z is n-pentyl, preferably, it is substituted by 4 OH groups.

Preferably, R¹ is a straight or branched chain alkyl group of 1, 2 or 3 carbon atoms optionally substituted with 1 or 2 groups independently selected from OH, CO₂H, CONH₂, OR², CO₂R² and CONHR², more preferably, OH and CO₂H.

Preferably, R² is a straight or branched chain alkyl group of 1, 2 or 3 carbon atoms optionally substituted with 1 or 2 groups independently selected from OH, CO₂H, CONH₂, OR³, CO₂R³ and CONHR³, more preferably, OH and CO₂H.

Preferably, R³ is a straight or branched chain alkyl group of 1, 2 or 3 carbon atoms.

Preferred compounds of the present invention are:

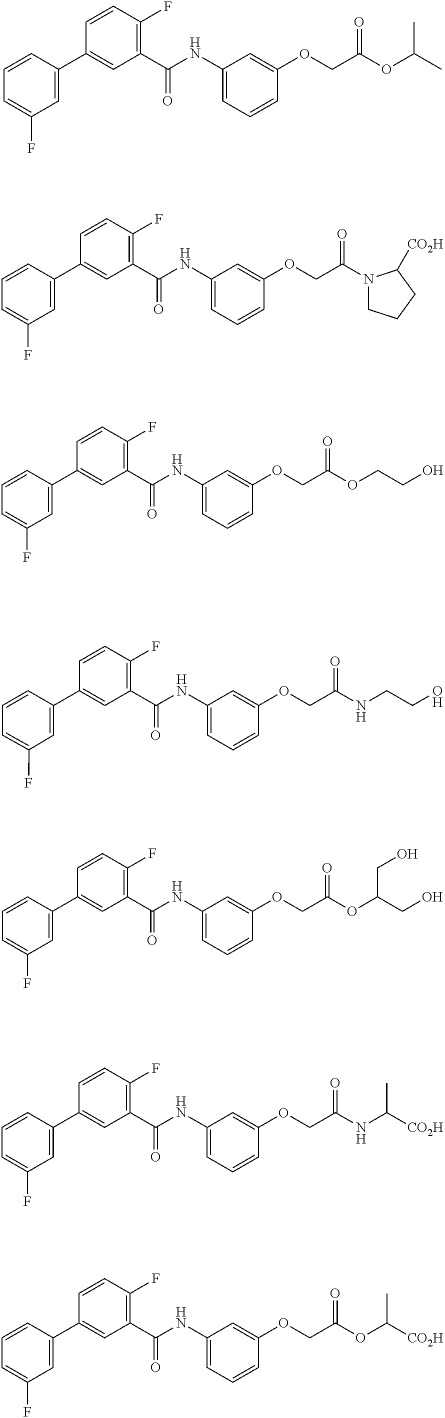

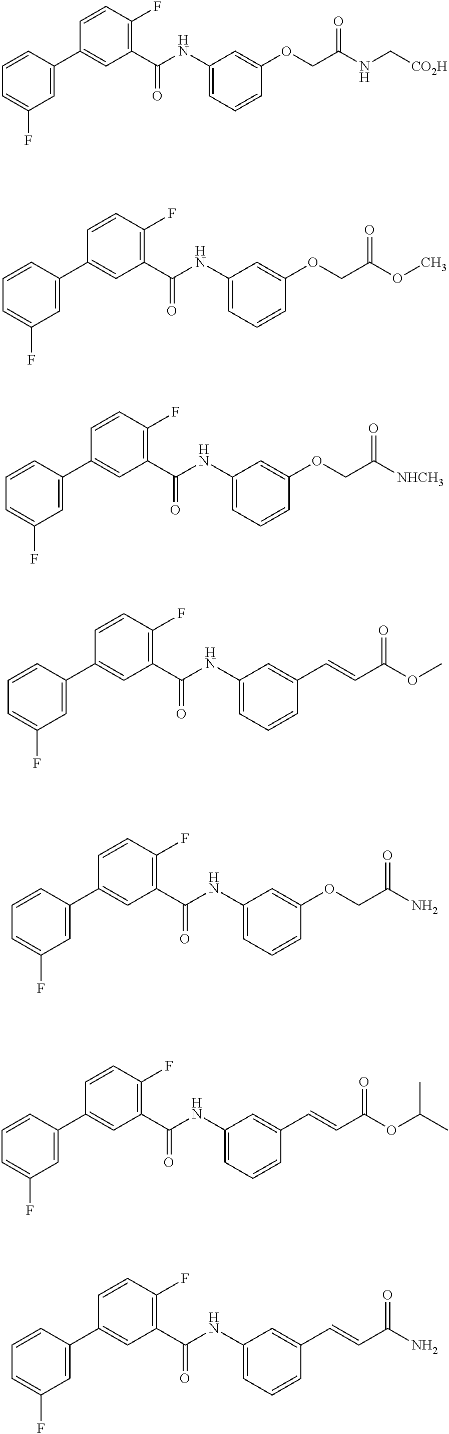

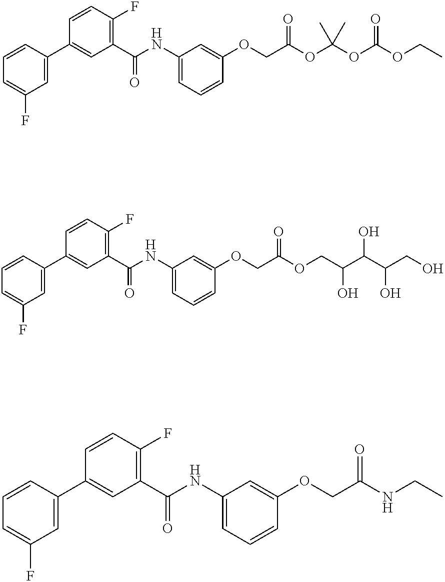

or a pharmaceutically acceptable salt or solvate thereof (where this is possible).

In some embodiments of the invention, the compound is selected from:

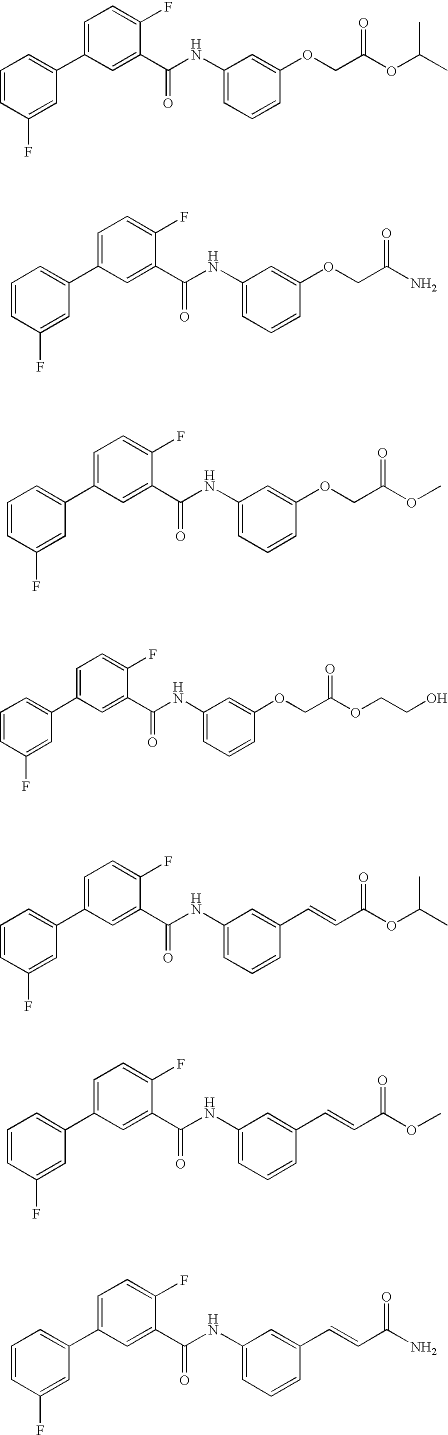

In a particular embodiment of the invention, the compound is:

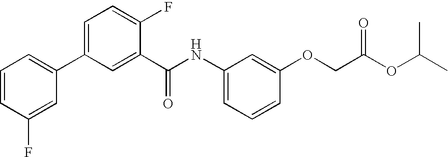

An eighth aspect of the invention provides a compound of formula (2):

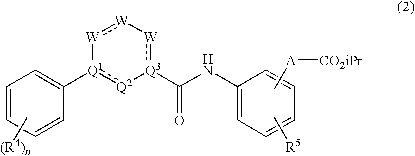

or a pharmaceutically acceptable salt or solvate wherein:
R⁴ is independently selected from the group consisting of H, halogen, methyl, methoxy, hydroxy, trifluoromethyl and trifluoromethoxy;
R⁵ is independently selected from the group consisting of H, halogen, methyl, methoxy, hydroxy, trifluoromethyl and trifluoromethoxy;
Q² is selected from a group consisting of CH, N and O;
each of Q¹ and Q³ can be independently a carbon or nitrogen atom;
each of W can be independently selected from a group consisting of (CR)₀₋₁, N, O and S where R is independently selected from the group consisting of H, halogen, methyl, methoxy, hydroxy, trifluoromethyl and trifluoromethoxy;
n=1, 2 or 3; and

### A=CH₂, CH₂CH₂, CH═CH or OCH₂.

A ninth aspect of the invention comprises a compound of formula (2) or a pharmaceutically acceptable salt or solvate thereof, as defined in the eighth aspect for use in a method of therapy.

A tenth aspect of the invention comprises a compound of formula (2) or a pharmaceutically acceptable salt or solvate thereof, as defined in the eighth aspect for use in the treatment of ocular hypertension.

An eleventh aspect of the present invention provides a pharmaceutical composition comprising a compound of formula (2) or a pharmaceutically active acceptable salt or solvate thereof, as defined in the eighth aspect together with a pharmaceutically acceptable carrier.

A twelfth aspect of the present invention provides a method of treating ocular hypertension which comprises administering to a mammal having ocular hypertension a therapeutically effective amount of a compound of the formula (2) or a pharmaceutically acceptable salt or solvate thereof as defined in the eighth aspect.

A thirteenth aspect of the present invention provides a method of providing neuroprotection to the eye of a mammal which comprises administering to a mammal in need of neuroprotection a therapeutically effective amount of a compound of the formula (2) or a pharmaceutically acceptable salt or solvate thereof as defined in the eighth aspect.

A fourteenth aspect of the present invention provides a contact lens or a contact lens solution comprising a compound of the formula (2) or a pharmaceutically acceptable salt or solvate thereof as defined in the eighth aspect.

### Further Features of the Eighth to Fourteenth Aspects of the Invention

Preferably Q¹ and Q³ are carbon atoms.

Preferably W═(CR)₀₋₁ or S.

Preferably R and R⁴ are H or F, most preferably F.

In some embodiments, —W—W—W— comprises two or three ring atoms. In these embodiments, it is preferred that either only one of the ring atoms is N, O or S or that all of the ring atoms are carbon.

In the embodiments where —W—W—W— comprises two or three ring atoms, and at least one ring atom is carbon, it is preferred that either only one of the carbon ring atoms bears a fluoro substituent or that none of the carbon ring atoms bears a fluoro substituent.

A is preferably OCH₂.

R⁵ is preferably H or F, preferably H.

R⁴ is preferably located in the meta position.

Preferably, the compound of formula (2) has the structure (2a):

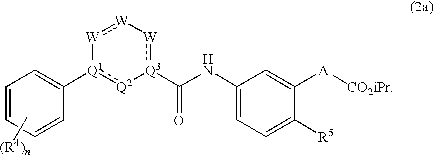

More preferably, the compound of formula (2) has the structure (2b):

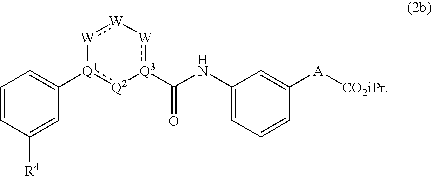

Most preferably, the compound of formula (2) has one of the following structures (2c or 2d)

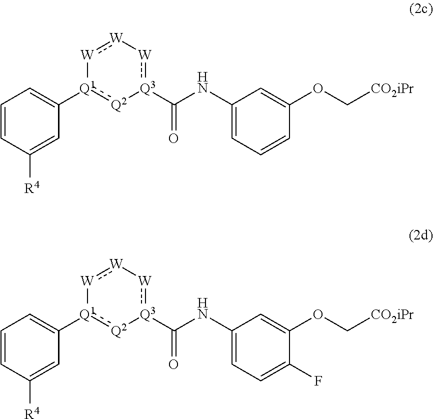

The group

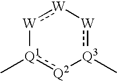

is an aromatic group. The bond [symbol] indicates either a double or single bond, as long as the group is aromatic. Preferably, the group is selected from phenylene, fluorophenylene, furanylene and pyridylene groups. The group may also be thiazolylene. Most preferably, the group

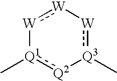

has a structure selected from:

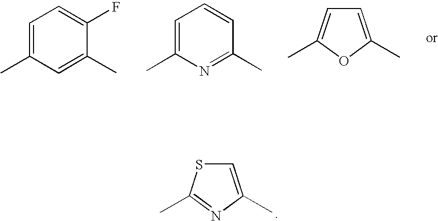

Particularly preferred compounds of the eighth to fourteenth aspects of the present invention include:

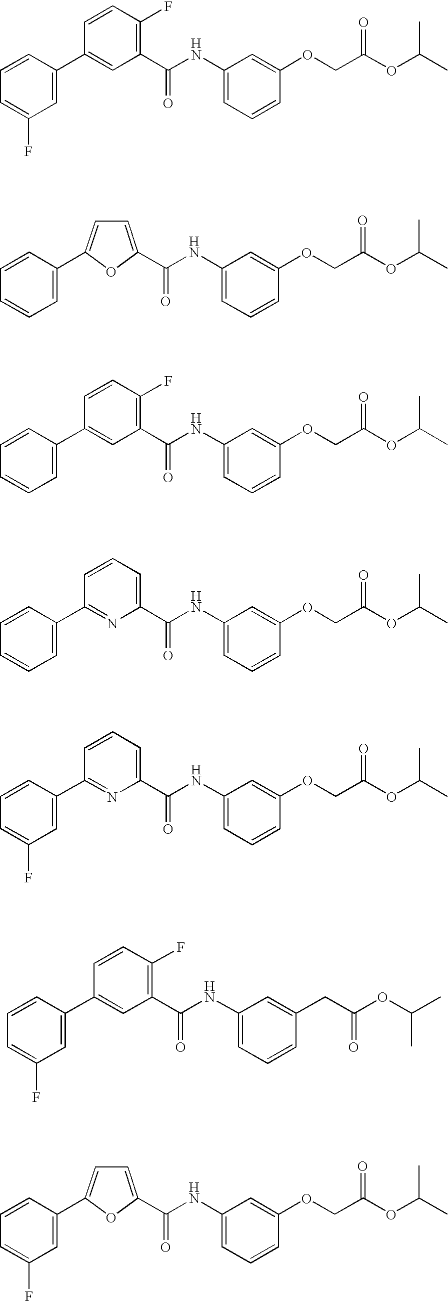

and pharmaceutically acceptable solvates.

A most preferred compound of the eighth to fourteenth aspects of the present invention is:

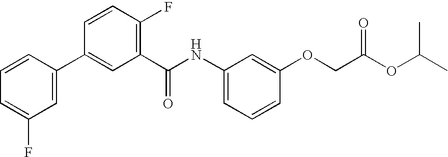

and pharmaceutically acceptable salts, solvates and chemically protected forms thereof.

### BRIEF DESCRIPTION OF THE DRAWINGS

FIG. 1. Effect on intraocular pressure (IOP) in the monkey following the topical administration of a single dose of 3-[(3′-Fluoro-4-fluorobiphenyl-3-carbonyl)amino]phenoxyacetic acid isopropyl ester (compound 2) at a concentration of 0.006% (w/v).

FIG. 2. IOP change from baseline in the cynomolgus monkey. 3-[(3′-Fluoro-4-fluorobiphenyl-3-carbonyl)amino]phenoxyacetic acid, isopropyl ester (compound 2) at 0.006%.

FIG. 3. IOP change from baseline in the cynomolgus monkey. 3-[(3′-fluoro-4-fluorobiphenyl-3-carbonyl)amino]phenoxyacetic acid (compound C1) at 0.01%.

FIG. 4. IOP change from baseline in the cynomolgus monkey. 3-[(3′-fluoro-4-fluorobiphenyl-3-carbonyl)amino]phenoxyacetic acid methyl ester (compound 4) at 0.01%.

FIG. 5. IOP change from baseline in the cynomolgus monkey. 3-[(3′-fluoro-4-fluorobiphenyl-3-carbonyl)amino]phenoxyacetamide (compound 3) at 0.003%.

FIG. 6. IOP change from baseline in the cynomolgus monkey. 3-[(3′-fluoro-4-fluorobiphenyl-3-carbonyl)amino]phenoxyacetic acid ethylene glycol ester (compound 1) at 0.01%.

FIG. 7. IOP change from baseline in the cynomolgus monkey. 3-[(3′-Fluoro-4-fluorobiphenyl-3-carbonyl)amino]cinnamic acid (compound C2) at 0.01%.

FIG. 8 shows the IOP change from baseline in beagle dogs following treatment by compound C1 and compounds of the present invention.

FIG. 9 shows the ocular surface hyperemia score in beagle dogs from treatment by compound C1 and compounds of the present invention.

### DETAILED DESCRIPTION OF THE INVENTION

The terms “comprising” and “comprises” means “including” as well as “consisting” e.g. a composition “comprising” X may consist exclusively of X or may include something additional e.g. X+Y.

“Optional” or “optionally” means that the subsequently described event of circumstances may or may not occur, and that the description includes instances where said event or circumstance occurs and instances in which it does not.

“May” means that the subsequently described event of circumstances may or may not occur, and that the description includes instances where said event or circumstance occurs and instances in which it does not.

As used herein, the term “substituted” is contemplated to include all permissible substituents of organic compounds as defined herein. The permissible substituents can be one or more and the same or different for appropriate organic compounds as defined herein. For purposes of this invention, the heteroatoms such as nitrogen may have hydrogen substituents and/or any permissible substituents of organic compounds described herein which satisfy the valencies of the heteroatoms.

A pharmaceutically acceptable salt is any salt which retains the activity of the parent compound and does not impart any deleterious or undesirable effect on the subject to whom it is administered in the context in which it is administered. Examples of pharmaceutically acceptable salts are discussed in Berge, et al., J. Pharm. Sci., 66, 1-19 (1977). Particularly preferred salts include those formed with inorganic ions, such as sodium, potassium, calcium, magnesium and zinc (Na⁺, K⁺, Ca²⁺ Mg²⁺ and Zn²⁺).

Additionally or alternatively, organic cations may be used to form salts. Examples include, but are not limited to, ammonium ion (i.e. NH₄⁺) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

The term “treatment”, as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

As used herein, ocular hypertension includes but is not limited to glaucoma.

Pharmaceutical compositions may be formulated for any suitable route and means of administration. Pharmaceutically acceptable carriers or diluents include those used in formulations suitable for oral, rectal, nasal, inhaled, topical (including ocular, buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients.

The compound of formulae (1) and (2) and pharmaceutically acceptable salts or solvates thereof are useful in lowering intraocular pressure and thus are useful in the treatment of ocular hypertension and/or glaucoma. The preferred route of administration is topical. The dosage range for topical administration is generally between about 0.0001 and about 1000 micrograms per eye (μg/eye) and is preferably between about 0.0005 and about 10 μg/eye and most preferably between about 0.001 and 1 μg/eye. The compounds of the present invention can be administered as solutions, suspensions, or emulsions (dispersions) in a suitable ophthalmic vehicle.

For ophthalmic application, preferably solutions or suspensions are prepared using a physiological saline solution as a major vehicle. The pH of such ophthalmic solutions or suspensions should preferably be maintained between 4.5 and 8.0, preferably with an appropriate buffer system. A neutral pH is preferred, but not essential.

The therapeutically-effective amount topically is between about 0.0001 and 5% (w/v) in liquid formulations, preferably about 0.001 to about 1% (w/v), more preferably about 0.003 and about 0.03 wt %. While the precise regimen is left to the discretion of the clinician, it is recommended that the resulting solution be topically applied by placing one or two drops drop in each eye from once-a-week to one or two times a day.

The term “therapeutically-effective amount”, as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen.

Other ingredients which may be desirable to use in the ophthalmic preparations of the present invention include pharmaceutically-acceptable preservatives, co-solvents, viscosity building agents, stabilizers, surfactants and other additives.

Ophthalmic products are typically packaged in multidose form, which generally require the addition of preservatives to prevent microbial contamination during use. Suitable preservatives include: benzalkonium chloride, thimerosal, chlorobutanol, methyl paraben, propyl paraben, phenylethyl alcohol, edetate disodium, sorbic acid, or other agents known to those skilled in the art. Such preservatives are typically employed at a concentration between about 0.001% and about 1.0% by weight.

Prostaglandins, and particularly ester derivatives, typically have limited solubility in water and therefore may require a surfactant or other appropriate co-solvent in the composition. Such co-solvents include: Polysorbate 20, 60 and 80; Pluronic™ F-68, F-84 and P-103; Tyloxapol™; Cremophor™ EL, sodium dodecyl sulfate; glycerol; PEG 400; propylene glycol; cyclodextrins; or other agents known to those skilled in the art. Such co-solvents are typically employed at a concentration between about 0.01% and about 2% by weight. These surfactants can be used solely or in combination. Preferred examples of the nonionic surfactants are polysorbate 80 [poly(oxyethylene)sorbitan monooleate] and polyoxyethylene hydrogenated castor oil 60, which are widely used as additives of ophthalmic solutions. A particularly preferred surfactant is polysorbate 80 (Tween 80-poly(oxyethylene)sorbitan monooleate).

Viscosity greater than that of simple aqueous solutions may be desirable to increase ocular absorption of the active compound, to decrease variability in dispensing the formulations, to decrease physical separation of components of a suspension or emulsion of formulation and/or otherwise to improve the ophthalmic formulation. Such viscosity building agents include, for example, polyvinyl alcohol, polyvinyl pyrrolidone, methylcellulose, hydroxypropylmethylcellulose, hydroxyethylcellulose, carboxymethylcellulose, hydroxypropylcellulose or other agents known to those skilled in the art. Such agents are typically employed at a concentration between about 0.01% and about 2% by weight.

To achieve a pH in the range between 4.5 and 8.0 and to maintain the pH for optimal stability during the shelf life of the composition, a buffer is often included in the ophthalmic solutions of the present invention.

Accordingly, preferred buffers include acetate buffers, citrate buffers, phosphate buffers and borate buffers. Acids or bases may be used to adjust the pH of these formulations as needed. However, borate is a particularly preferred buffer for use in ophthalmic compositions, since it has some inherent antimicrobial activity and often enhances the activity of antimicrobials or other buffers.

As used herein, the term “borate” shall refer to boric acid, salts of boric acid and other pharmaceutically acceptable borates, or combinations thereof. Most suitable are: boric acid, sodium borate, potassium borate, calcium borate, magnesium borate, manganese borate, and other such borate salts.

Preferred carriers which may be used in the ophthalmic preparations of the present invention include, but are not limited to, polyvinyl alcohol, povidone, hydroxypropyl methyl cellulose, poloxamers, carboxymethyl cellulose, carbomers, hydroxyethyl cellulose, cyclodextrin and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

An ophthalmically acceptable antioxidant may also be added in order to prevent the concentration of the compound of formula (1) or (2) (or a pharmaceutically acceptable salt or solvate thereof) of the present invention from lowering by inhibiting decomposition of the compound of formula (1) or (2) in an ophthalmic solution. Specific examples of antioxidants are sodium nitrite, ascorbic acid, L-ascorbic acid stearate, sodium hydrogensulfite, sodium metabisulfite, sodium thiosulfate, thiourea, acetylcysteine, butylated hydroxyanisole, butylated hydroxytoluene, alphathioglycerin, ethylenediaminetetraacetic acid, erythorbic acid, cysteine hydrochloride, citric acid, tocopherol acetate, potassium dichloroisocyanurate, soybean lecithin, sodium thioglycollate, sodium thiomalate, natural vitamin E, tocopherol, ascorbyl pasthyminate, sodium pyrosulfite, 1,3-butylene glycol, pentaerythtyl tetrakis[3-(3,5-di-t-butyl-4-hydroxyphenyl)]propionate, propyl gallate, 2-mercaptobenzimidazole and oxyquinoline sulfate. These antioxidants can be used solely or in combination.

Preferred examples of antioxidants are ethylenediaminetetraacetic acid, salts thereof and dibutylhydroxytoluene, which are widely used as additives of ophthalmic solutions. It is particularly preferable to combine ethylenediaminetetraacetic acid or the salt thereof with dibutylhydroxytoluene.

The ingredients of the ophthalmic compositions of the present invention are preferably included in the following amounts:

| Ingredient | Amount (% w/v) |
|---|---|
| Active ingredient | About 0.0001-5.0 |
| Preservative | 0-0.10 |
| Vehicle | 0-40 |
| Tonicity adjustor | 0-10 |
| Buffer | 0.0-10 |
| pH adjustor | q.a. pH 4.5-8.0 |
| Antioxidant | As needed |
| Surfactant | As needed |
| Purified water | As needed to make 100% |

The ophthalmic formulations for use in the method of the present invention are conveniently packaged in forms suitable for metered application, such as in containers equipped with a dropper, to facilitate application to the eye. Containers suitable for dropwise application are usually made of suitable inert, non-toxic plastic material, and generally contain between about 0.5 and about 15 ml solution. One package may contain one or more unit doses.

Especially preservative-free solutions are often formulated in non-resealable containers containing up to about ten, preferably up to about five unit doses, where a typical unit dose is from one to about 8 drops, preferably one to about 3 drops. The volume of one drop usually is about 20-35 μl.

At a concentration of 0.01% w/v or lower, the compound of the present invention preferably depresses the intraocular pressure by greater than 5 mmHg, more preferably greater than 10 mmHg, more preferably greater than 20 mmHg, more preferably greater than 30 mmHg compared to pharmaceutically acceptable carrier (such as 1% polysorbate 80 in 5 nM Tris HCl).

Preferably, a concentration of 0.01% w/v or lower, the compounds of the present invention preferably depresses the intraocular pressure by greater than 10 mmHg for greater than 12 hours, more preferably greater than or equal to 24 hours, more preferably greater than or equal to 48 hours, for example, up to 72 hours compared to pharmaceutically acceptable carrier.

At a concentration of between 0.008% w/v and 0.004% w/v, more preferably 0.007% w/v and 0.005% w/v, most preferably at about 0.006% w/v, the compounds of the present invention preferably depresses the intraocular pressure by greater than 5 mmHg, more preferably greater than 10 mmHg, more preferably greater than 20 mmHg, more preferably greater than 30 mmHg, compared to pharmaceutically acceptable carrier.

Preferably, a concentration of between 0.008% w/v and 0.004% w/v, more preferably 0.007% w/v and 0.005% w/v, most preferably at about 0.006% w/v, the compounds of the present invention preferably depresses the intraocular pressure by greater than 10 mmHg, more preferably greater than 20 mmHg, more preferably greater than 30 mmHg, for greater than 12 hours, more preferably greater than or equal to 24 hours, more preferably greater than or equal to 48 hours, for example, up to 72 hours compared to pharmaceutically acceptable carrier.

The compounds described herein are either the pharmacological active, or are a prodrug of a pharmacological active. The term “prodrug” as used throughout this text means the pharmacologically acceptable derivatives such as esters and amides, such that the resulting in vivo biotransformation product of the derivative is the active drug. The reference by Goodman and Gilman (The Pharmacological Basis of Therapeutics, 8th ed., McGraw-HiM, Int. Ed. 1992, “Biotransformation of Drugs”, p 13-15) describing prodrugs generally is hereby incorporated.

The term stereochemically isomeric forms of compounds of the present invention, as used hereinbefore, defines all possible compounds made up of the same atoms bonded by the same sequence of bonds but having different three-dimensional structures which are not interchangeable, which the compounds of the present invention may possess.

Unless otherwise mentioned or indicated, the chemical designation of a compound encompasses the mixture of all possible stereochemically isomeric forms which said compound may possess. Said mixture may contain all diastereomers and/or enantiomers of the basic molecular structure of said compound. All stereochemically isomeric forms of the compounds of the present invention both in pure form or in admixture (for example racemic mixtures) with each other are intended to be embraced within the scope of the present invention.

Pure stereoisomeric forms of the compounds and intermediates as mentioned herein are defined as isomers substantially free of other enantiomeric or diastereomeric forms of the same basic molecular structure of said compounds or intermediates. In particular, the term ‘stereoisomerically pure’ concerns compounds or intermediates having a stereoisomeric excess of at least 80% (i.e. minimum 90% of one isomer and maximum 10% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stereoisomeric excess of 97% up to 100%. The terms ‘enantiomerically pure’ and ‘diastereomerically pure’ should be understood in a similar way, but then having regard to the enantiomeric excess, respectively the diastereomeric excess of the mixture in question.

The compound of formula (1) or (2) and the preferred compounds referred to herein are intended to include stereoisomerically pure, enantiomerically pure and diastereomerically pure compounds and compositions where these possibilities exist.

Pure stereoisomeric forms of the compounds and intermediates of this invention may be obtained by the application of art-known procedures. For instance, enantiomers may be separated from each other by the selective crystallization of their diastereomeric salts with optically active acids. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound will be synthesized by stereospecific methods of preparation. These methods will advantageously employ enantiomerically pure starting materials.

As used herein, the term “on the backbone” when referring to a substitution, means that one or more hydrogen atoms on the backbone is replaced by one or more of the groups indicated. Where more than one substitution occurs, they may be on the same, adjacent or remote carbon atoms, i.e., located on carbon atoms that are 0, 1, 2, 3, 4 or 5 carbon atoms apart.

Where a group comprises two or more moieties defined by a single carbon atom number, for example, C₂₋₅ alkyl, the carbon atom number indicates the total number of carbon atoms in the group.

As used herein, the term “alkyl” refers to a straight or branched saturated monovalent hydrocarbon radical, having the number of carbon atoms as indicated. By way of non limiting example, suitable alkyl groups include methyl, ethyl, propyl, butyl and pentyl.

The invention is further illustrated by the following examples which are illustrative of a specific mode of practicing the invention and are not intended as limiting the scope of the claims.

### Example 1

### 3-[(3′-Fluoro-4-fluorobiphenyl-3-carbonyl)amino]phenoxyacetic acid, ethylene glycol ester (Compound 1)

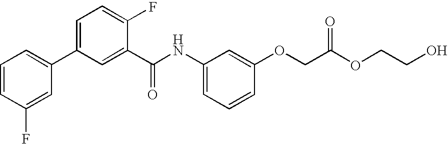

To a solution of 3-[(3′-fluoro-4-fluorobiphenyl-3-carbonyl)amino]phenoxyacetic acid (80 mg), ethylene glycol (0.1 ml) and 4-dimethylaminopyridine (13 mg) in chloroform (amylene stabilized; 2 ml) was added EDC hydrochloride (50 mg). The resulting mixture was stirred at ambient temperature for 16 hours, diluted with ethyl acetate (25 ml), washed twice with 2M hydrochloric acid then with sodium carbonate solution and brine. After drying the organic extract over sodium sulphate, the solvent was removed in vacuo. The title compound (35 mg; m.p. 135-137° C.) was obtained as a white solid following silica gel chromatography of the residue in 1:1 petroleum ether (b.p. 40-60° C.):ethyl acetate then re-crystallisation from ethyl acetate/petroleum ether (b.p. 40-60° C.).

¹H NMR (d⁶-DMSO, δ): 3.6 (2H, m); 4.15 (2H, t); 4.8 (2H, s); 4.9 (1H, t): 6.7 (1H, m); 7.2-7.6 (8H, c); 7.9 (2H, c); 10.5 (1H, s). Mass Spectrum (m/z) ES−: 426.1 (M−H)⁻

### Example 2

### 3-[(3′-Fluoro-4-fluorobiphenyl-3-carbonyl)amino]phenoxyacetic acid, isopropyl ester (Compound 2)

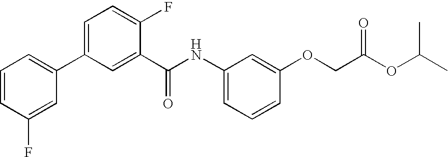

To a solution of 3-[(3′-fluoro-4-fluorobiphenyl-3-carbonyl)amino]phenoxyacetic acid (150 mg), DMAP (25 mg) and isopropanol (0.1 ml) in DCM (2 ml) was added DCC (1M in DCM; 0.5 ml). After 1 hour the mixture was evaporated, slurried in ethyl acetate (20 ml) and filtered. The filtrate was washed with 2M hydrochloric acid, brine, dried over sodium sulphate and evaporated in vacuo. The title compound was obtained as a white solid following silica gel chromatography of the residue in 3:1 petroleum ether (b.p. 40-60° C.):ethyl acetate (61 mg; m.p. 110-111° C.).

¹H NMR (CDCl₃, δ): 1.3 (6H, d); 4.6 (2H, s); 5.2 (1H, septet); 6.8 (1H, m); 7.1-7.5 (8H, c); 7.7 (1H, m); 8.4 (1H, m); 8.5 (1H, br d). Mass Spectrum (m/z) ES−: 448.1 (M+Na)⁺

### Example 3

### 3-[(3′-Fluoro-4-fluorobiphenyl-3-carbonyl)amino]phenoxyacetamide (Compound 3)

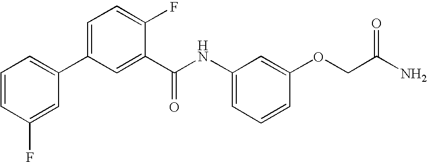

A solution of 3-[(3′-fluoro-4-fluorobiphenyl-3-carbonyl)amino]phenoxyacetic acid (150 mg) and 1,1′-carbonyldiimidazole (0.85 g) in THF (3.5 ml) and N,N-dimethylacetamide (1 ml) was stirred at room temperature for 1 hour. Aqueous ammonia (30%; 0.6 ml) was added and the resulting mixture was left for 18 hours. The mixture was concentrated in vacuo, ethyl acetate (5 ml) and water (5 ml) were added and the mixture stirred for 10 minutes. The resulting precipitate was filtered, washed with ethyl acetate and water and dried in vacuo to yield the title compound (65 mg; m.p. 203-206° C.). The filtrate was separated, the organic layer washed with brine, dried over sodium sulphate and evaporated in vacuo. Trituration of the residue in DCM/pentane yielded further title compound (85 mg).

¹H NMR (d⁶-DMSO, δ): 4.4 (2H, s); 6.7 (1H, m); 7.2-7.6 (10H, c); 7.9 (2H, c); 10.5 (1H, s). Mass Spectrum (m/z) ES+: 405.2 (M+Na)⁺

### Example 4

### 3-[(3′-Fluoro-4-fluorobiphenyl-3-carbonyl)amino]phenoxyacetic acid, methyl ester (Compound 4)

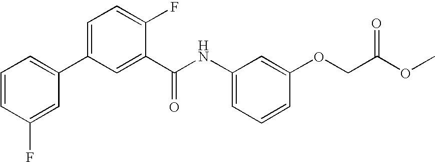

3-[(3′-Fluoro-4-fluorobiphenyl-3-carbonyl)amino]phenoxyacetic acid (125 mg) and 4-toluenesulphonic acid (1 mg) in anhydrous methanol (10 ml) were heated to reflux for 16 hours. The mixture was cooled, evaporated in vacuo and the residue chromatographed on silica gel in 2:1 petroleum ether (b.p. 40-60° C.):ethyl acetate. The resultant solid was re-crystallised from DCM/pentane to yield the title product (94 mg; m.p. 103-104° C.).

¹H NMR (CDCl3, δ): 3.7 (3H, s); 4.6 (2H, s); 6.8 (1H, m); 7.1-7.5 (8H, c); 7.7 (1H, m); 8.4 (1H, m); 8.5 (1H, br d). Mass Spectrum (m/z) ES+: 420.2 (M+Na)⁺

### Example 5

### 3-[(3′-Fluoro-4-fluorobiphenyl-3-carbonyl)amino]cinnamic acid, isopropyl ester (Compound

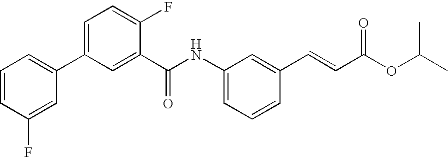

To a solution of 3-[(3′-fluoro-4-fluorobiphenyl-3-carbonyl)amino]cinnamic acid (150 mg), DMAP (25 mg) and isopropanol (0.1 ml) in DCM (2 ml) was added DCC (1M in DCM; 0.5 ml).

After 1 hour the mixture was evaporated, slurried in ethyl acetate (20 ml) and filtered. The filtrate was washed with 2M hydrochloric acid, brine, dried over sodium sulphate and evaporated in vacuo. The title compound was obtained as a white solid following silica gel chromatography of the residue in 3:1 petroleum ether (b.p. 40-60° C.):ethyl acetate (76 mg; m.p. 154-155° C.).

¹H NMR (CDCl₃, δ): 1.3 (6H, d); 5.2 (1H, septet); 6.5 (2H, d); 7.1 (1H, m); 7.3-7.7 (8H, c); 8.0 (1H, s); 8.4 (1H, m); 8.6 (1H, br d). Mass Spectrum (m/z) ES+: 422.1 (M+H)⁺

### Example 6

### 3-[(3′-Fluoro-4-fluorobiphenyl-3-carbonyl)amino]cinnamic acid, methyl ester (Compound 6)

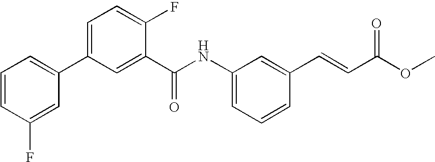

3-[(3′-Fluoro-4-fluorobiphenyl-3-carbonyl)amino]cinnamic acid (150 mg) and 4-toluenesulphonic acid (1 mg) in anhydrous methanol (10 ml) were heated to reflux for 16 hours. The mixture was cooled and filtered. The white solid was washed with methanol (2×5 ml) and dried in vacuo to yield the title compound (27 mg). The filtrate was evaporated and the residue chromatographed on silica gel in 2:1 petroleum ether (b.p. 40-60° C.):ethyl acetate. The resultant solid was triturated in DCM/petroleum ether (b.p. 40-60° C.) to yield further title compound (102 mg; m.p. 144-145° C.).

¹H NMR (CDCl3, δ): 3.8 (3H, s); 6.5 (2H, d); 7.1 (1H, m); 7.3-7.7 (8H, c); 7.9 (1H, s); 8.4 (1H, m); 8.6 (1H, br d). Mass Spectrum (m/z) ES+: 394.1 (M+H)⁺

### Example 7

### 3-[(3′-Fluoro-4-fluorobiphenyl-3-carbonyl)amino]cinnamamide (Compound 7)

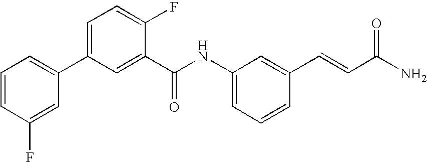

A solution of 3-[(3′-fluoro-4-fluorobiphenyl-3-carbonyl)amino]cinnamic acid (200 mg) and 1,1′-carbonyldiimidazole (0.85 g) in THF (5 ml) was stirred at room temperature for 2 hours. Aqueous ammonia (30%; 0.75 ml) was added and the resulting mixture was left for 4 hours. The mixture was partitioned between ethyl acetate (15 ml) and 2M hydrochloric acid (15 ml), the organic layer separated, washed with water, sodium carbonate solution, brine, dried over sodium sulphate and evaporated in vacuo. The residue was triturated in DCM then dissolved in 5% methanol in DCM, washed with 1M sodium hydroxide solution, brine, 1M hydrochloric acid, dried over sodium sulphate and evaporated in vacuo to afford the title compound (74 mg; m.p. 216-218° C.).

¹H NMR (d⁶-DMSO, δ): 6.6 (1H, d); 7.1-7.6 (11H, c); 7.9-8.1 (3H, c); 10.6 (1H, s). Mass Spectrum (m/z) ES+: 401.2 (M+Na)⁺

### Example 8

Cynomolgus monkeys (Macaca fascicularis) were used for the intraocular pressure studies. Each animal was unilaterally laser-treated by circumferential laser photocoagulation to induce ocular hypertension in one eye. Conscious female animals were trained to sit in custom designed chairs and to accept applanation pneumatonometry. The drug was administered topically to one eye using a dropper bottle to deliver approximately a 35 μl volume, the other eye received vehicle (1% polysorbate 80 in 5 mM Tris HCl) as a control.

Proparacaine at 0.25% was used for corneal anesthesia during tonometry. Intraocular pressure was determined just before drug administration and at 2, 4, 6 and 24 hours. In addition, in some experiments, intraocular pressure was also determined at 48, 55, 72, 90 and 115 hours.

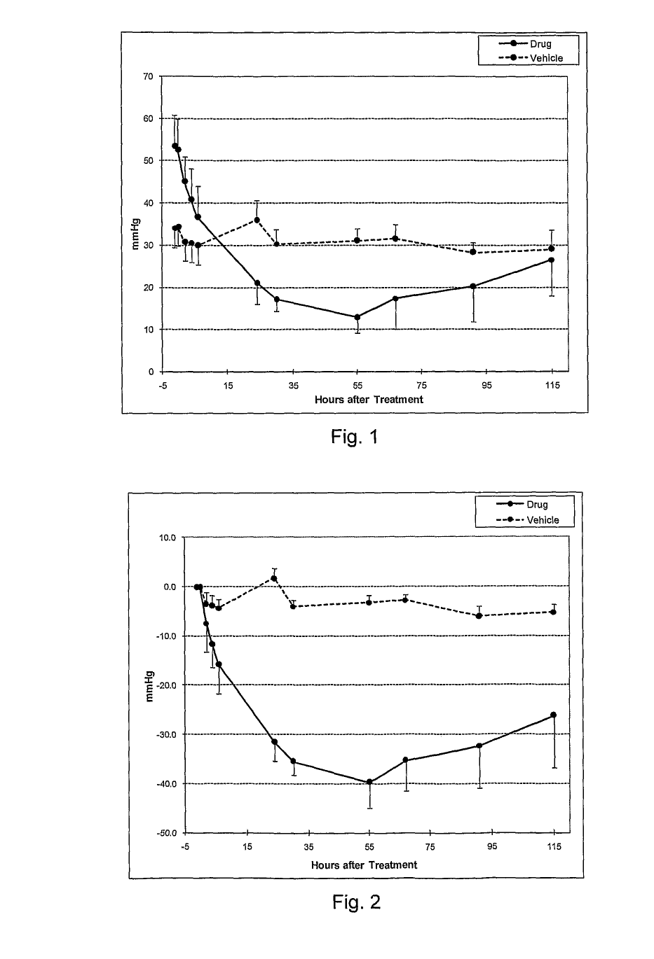

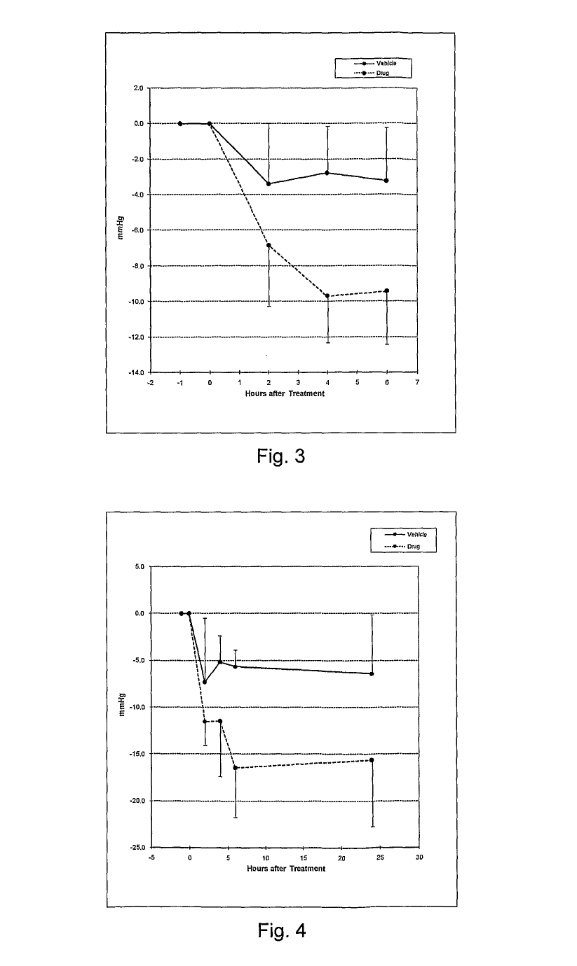

FIG. 1 shows the effect on intraocular pressure (IOP) in the monkey following the topical administration of a single dose of 3-[(3′-fluoro-4-fluorobiphenyl-3-carbonyl)amino]phenoxyacetic acid isopropyl ester (Compound 2) at a concentration of 0.006% (w/v) over 115 hours. FIG. 2 shows the IOP change from baseline over 6 hours in this test.

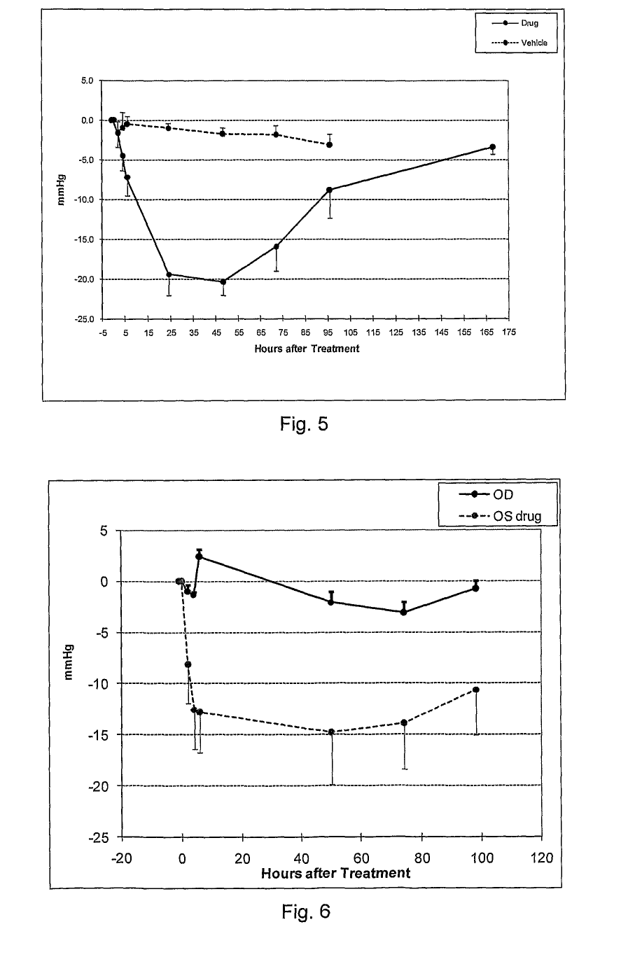

FIG. 3 shows the IOP change from baseline in the cynomolgus monkey following the topical administration of a single dose of 3-[(3′-fluoro-4-fluorobiphenyl-3-carbonyl)amino]phenoxyacetic acid (Compound C1) at 0.01%.

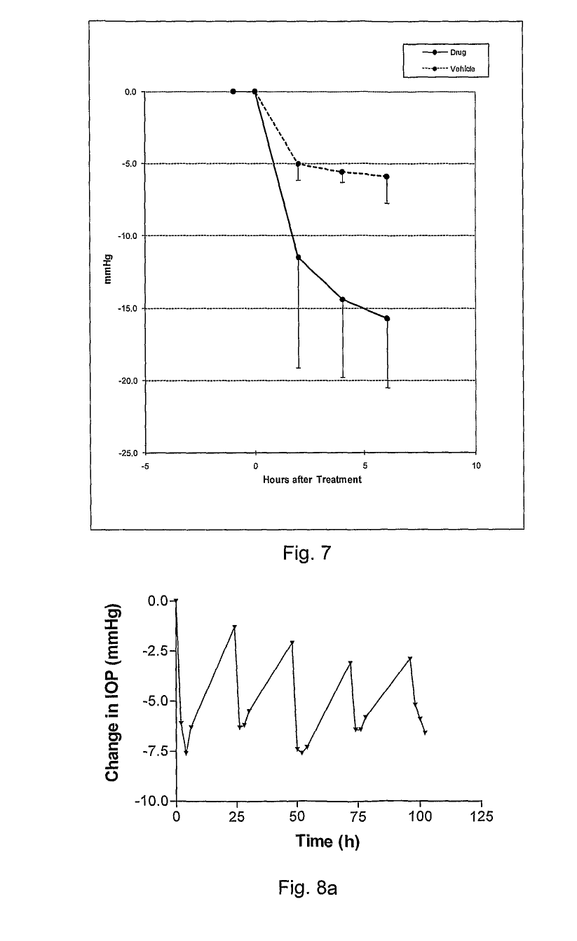

FIG. 4 shows the IOP change from baseline in the cynomolgus monkey following the topical administration of a single dose of 3-[(3′-fluoro-4-fluorobiphenyl-3-carbonyl)amino]phenoxyacetic acid methyl ester (Compound 4) at 0.01%.

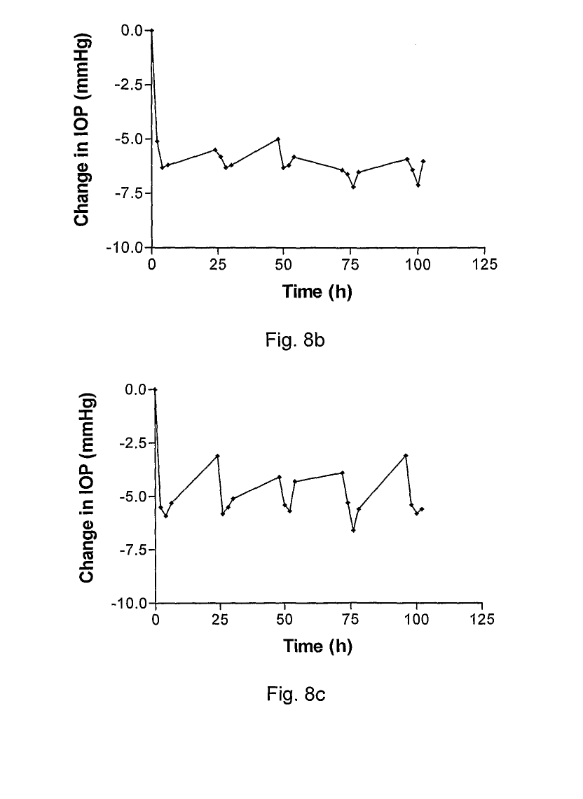

FIG. 5 shows the IOP change from baseline in the cynomolgus monkey following the topical administration of a single dose of 3-[(3′-fluoro-4-fluorobiphenyl-3-carbonyl)amino]phenoxyacetamide (Compound 3) at 0.003%.

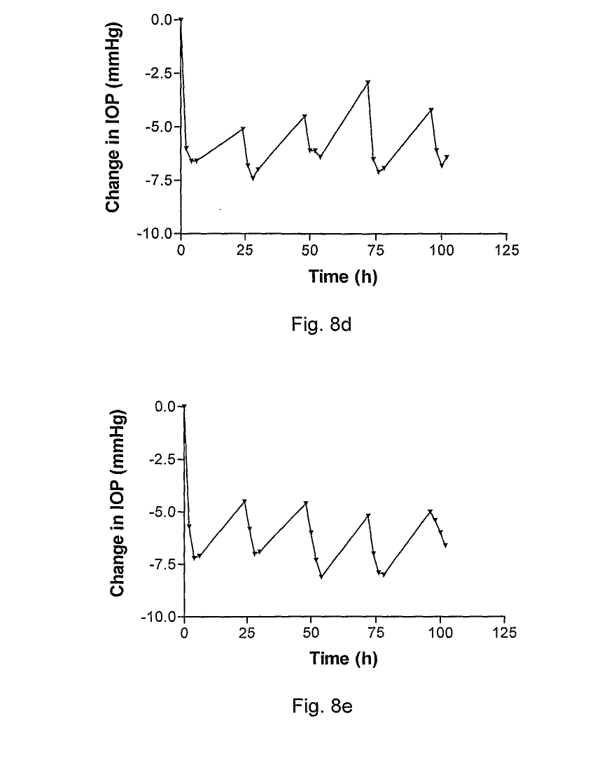

FIG. 6 shows the IOP change from baseline in the cynomolgus monkey following the topical administration of a single dose of 3-[(3′-fluoro-4-fluorobiphenyl-3-carbonyl)amino]phenoxyacetic acid ethylene glycol ester (Compound 1) at 0.01%.

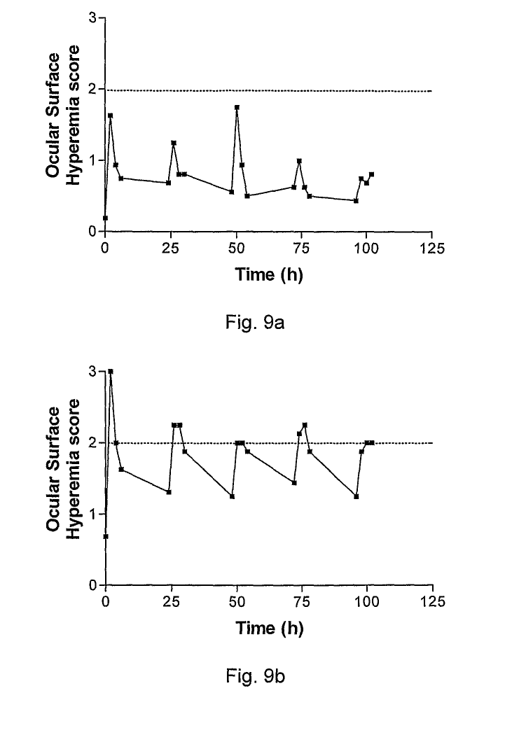

FIG. 7 shows the IOP change from baseline in the cynomolgus monkey following the topical administration of a single dose of 3-[(3′-Fluoro-4-fluorobiphenyl-3-carbonyl)amino]cinnamic acid (Compound C2) at 0.01%.

Measurement of intraocular pressure studies in dogs involved applanation pneumatonometry performed in Beagle dogs of both sexes. The animals remained conscious throughout the study and were gently restrained by hand. The drug was administered topically to the one eye using a dropper bottle to deliver approximately 35 μl volume, the other eye received vehicle (1% polysorbate 80 in 5 mM Tris HCl) as a control.

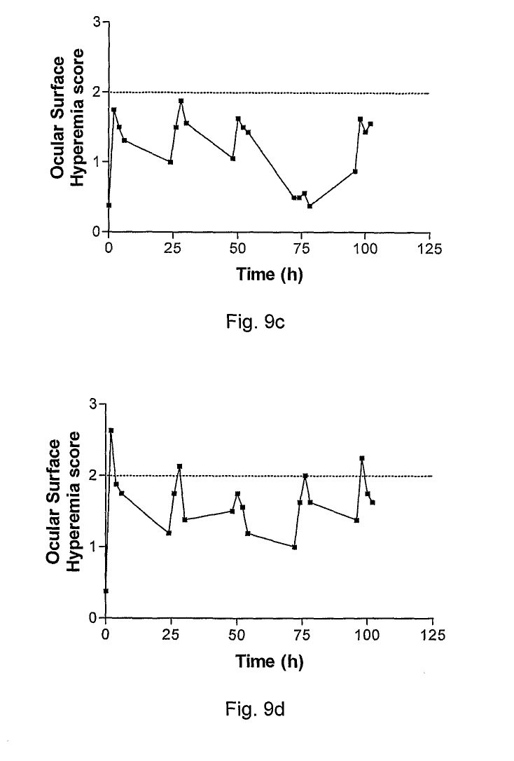

Proparacaine at 0.25% was used for corneal anesthesia during tonometry. Intraocular pressure was determined just before drug administration and at 2, 4, 6 hours thereafter on each day of the 5 day study. FIG. 8 show the results measured for the compounds tested as follows:

| FIG. | Compound |
|---|---|
| 8a | C1 |
| 8b | 1 |
| 8c | 4 |
| 8d | 2 |
| 8e | 3 |

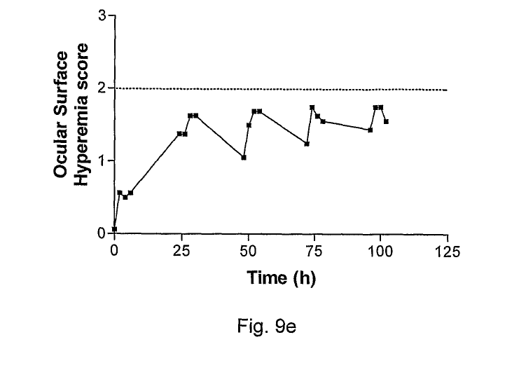

Measurement of ocular surface hyperemia was performed immediately before each of the intraocular pressure readings. Ocular surface hyperemia grading was semi-quantitative and assessed according to a 5 point scoring scale used for clinical evaluations: 0=none; 0.5=trace; 1=mild; 2=moderate; and 3=severe. FIG. 9 show the results measured for the compounds tested as follows:

| FIG. | Compound |
|---|---|
| 9a | C1 |
| 9b | 1 |
| 9c | 4 |
| 9d | 2 |
| 9e | 3 |

1. A method of treating an ocular hypertensive condition in a mammal or of providing neuroprotection to an eye of a mammal, comprising administering to a mammal in need thereof, a therapeutically effective amount of a compound of formula (1) 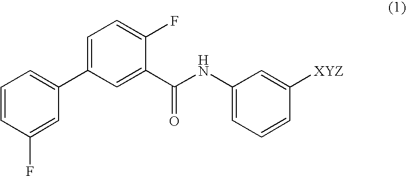
or a pharmaceutically acceptable salt or solvate thereof, wherein:
X is OCH₂, CH═CH or CH₂;
Y is —CO₂ or —C(O)NH;
Z is a straight or branched chain alkyl group of 1-6 carbon atoms, a cycloalkyl group of 1-6 carbon atoms, either of which may be optionally substituted with one or more groups selected from OH, CO₂H, CONH₂, OR¹, CO₂R¹, CONHR¹ and OCO₂R¹;
R¹ is a straight or branched chain alkyl group of 1-6 carbon atoms optionally substituted with one or more groups selected from OH, CO₂H, CONH₂, OR², CO₂R² and CONHR²;
R² is selected from a straight or branched chain alkyl group of 1-6 carbon atoms optionally substituted with one or more groups independently selected from OH, CO₂H, CONH₂, OR³, CO₂R³ and CONHR³; and
R³ is a straight or branched chain alkyl group of 1-6 carbon atoms; or
YZ together form a group selected from 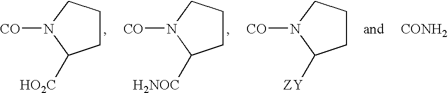
where Y and Z are as defined above.

2. The method according to claim 1, wherein X is OCH₂.

3. The method according to claim 1, wherein YZ is CONH₂.

4. The method according to claim 1, wherein Y is —CO₂.

5. The method according to claim 4, wherein Z is a straight or branched chain alkyl group having 1, 2 or 3 carbon atoms.

6. The method according to claim 4, wherein Z is selected from the group consisting of methyl, ethyl, n-propyl, iso-propyl, n-butyl, sec-butyl, iso-butyl and tert-butyl.

7. The method according to claim 4, wherein Z is substituted with 1 or 2 groups independently selected from the group consisting of OH and CO₂H.

8. The method according to claim 1, wherein the compound of formula (I) is selected from the group consisting of: 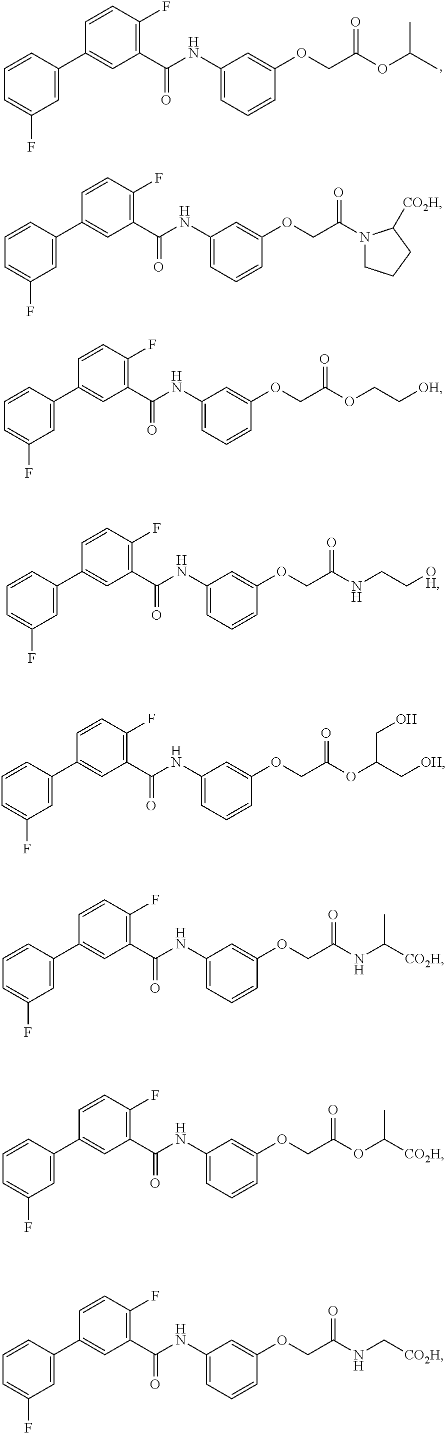 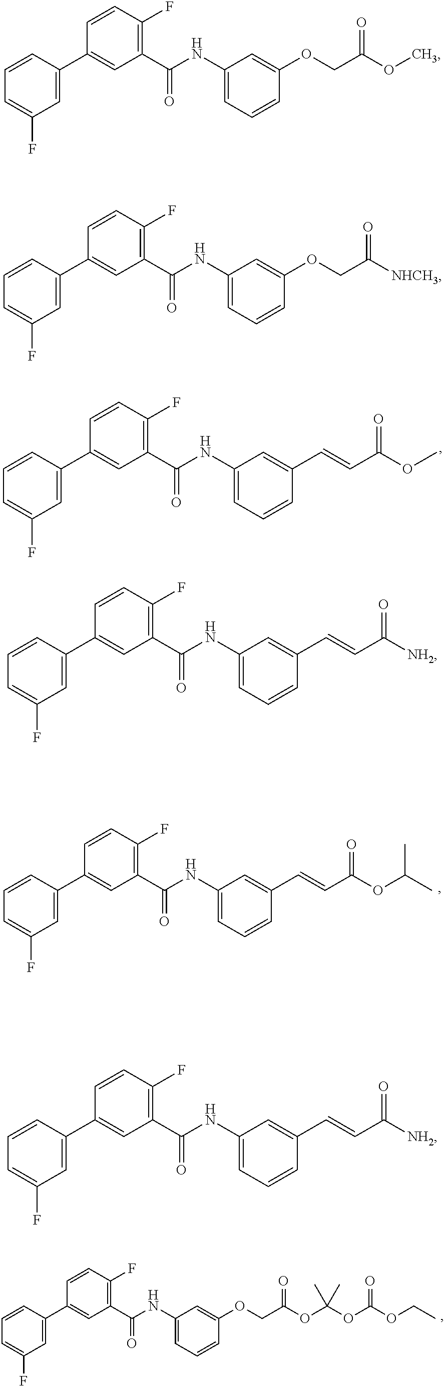 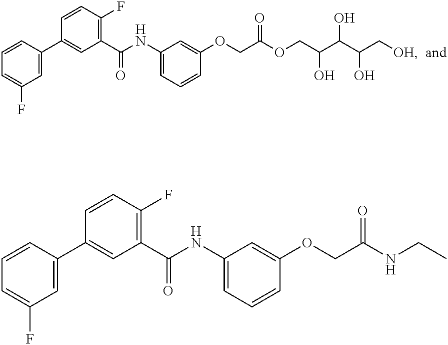
or a pharmaceutically acceptable salt or solvate thereof.

9. The method according to claim 1 wherein the compound is: 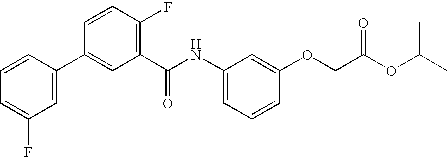
or a pharmaceutically acceptable salt or solvate thereof.

10. The method according to claim 1 wherein the compound is: 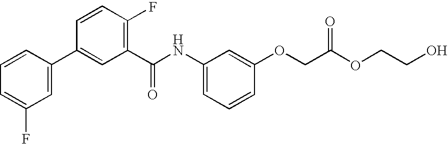
or a pharmaceutically acceptable solvate thereof.

11. A compound of formula (1) as defined in claim 1, or a pharmaceutically acceptable salt or solvate thereof, with the proviso that the compound does not have the structure:

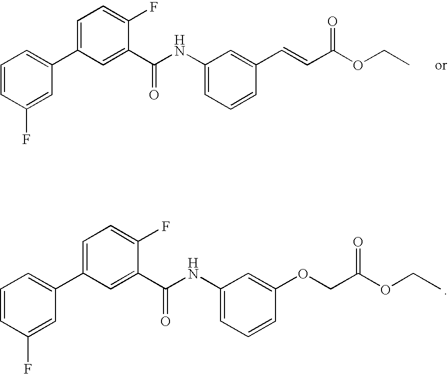

12. A compound of formula (2): 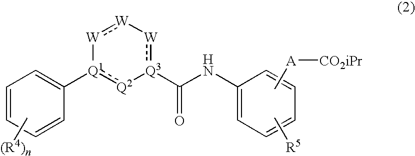
or a pharmaceutically acceptable salt or solvate wherein:
R⁴ is independently selected from the group consisting of H, halogen, methyl, methoxy, hydroxy, trifluoromethyl and trifluoromethoxy;
R⁵ is independently selected from the group consisting of H, halogen, methyl, methoxy, hydroxy, trifluoromethyl and trifluoromethoxy;
Q² is selected from a group consisting of CH, N and O;
each of Q¹ and Q³ is independently a carbon or nitrogen atom;
each of W is independently selected from the group consisting of (CR)₀₋₁, N, O and S where each R is independently selected from the group consisting of H, halogen, methyl, methoxy, hydroxy, trifluoromethyl and trifluoromethoxy;
n=1, 2 or 3; and
A=CH₂, CH₂CH₂, CH═CH or OCH₂.

13. The compound according to claim 12, wherein R⁵ is H or F.

14. The compound according to claim 12, wherein A is OCH₂.

15. The compound according to claim 12, wherein the compound is of formula (2a):

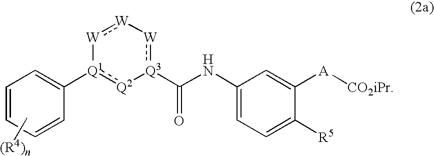

16. The compound according to claim 12, wherein the compound is of formula (2b):

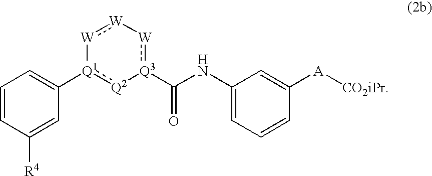

17. The compound according to claim 12, wherein the group 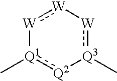
has the structure:

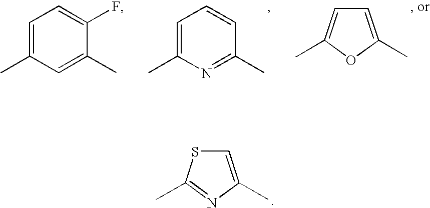

18. The compound according to claim 12 having the structure:

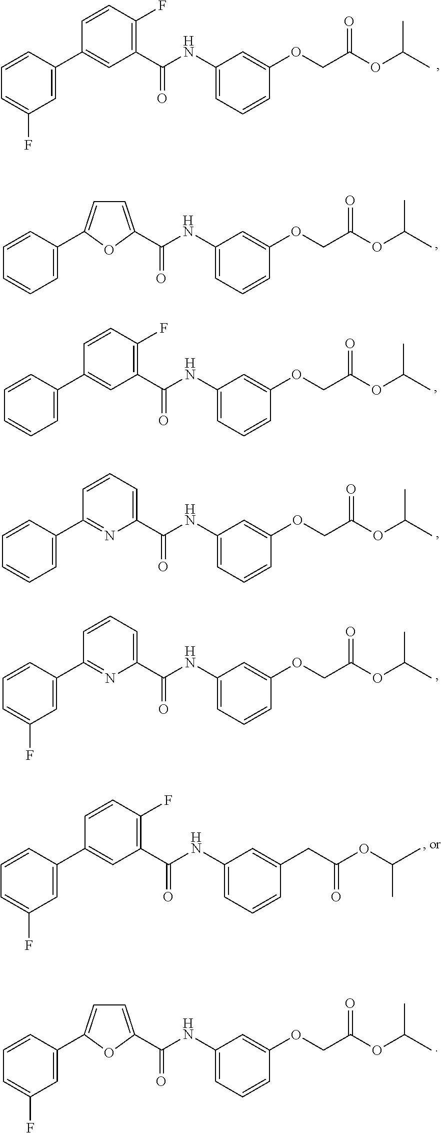

19-24. (canceled)

25. The method of claim 1, wherein the method is a method of treating an ocular hypertensive condition.

26. The method of claim 25, wherein the condition is glaucoma.

27. The method of claim 1, wherein the method is a method of providing neuroprotection to an eye of a mammal.

28. A pharmaceutical composition comprising a compound as defined in claim 1, and a pharmaceutically acceptable carrier, diluent, preservative, buffer or antioxidant.

29. A pharmaceutical composition comprising a compound according to claim 12, and a pharmaceutically acceptable carrier, diluent, preservative, buffer or antioxidant.

30. A contact lens or a contact lens solution comprising a compound as defined in claim 1.

31. A contact lens or a contact lens solution comprising a compound according to claim 12.
